(19) United States
(12) Patent Application Publication
(10) Pub. No.: US 2016/0143620 A1
(43) Pub. Date: May 26, 2016

(54) ASSESSMENT ASSISTANCE DEVICE

(71) Applicant: FUJIFILM Corporation, Tokyo (JP)

(72) Inventors: Yasunori OHTA, Ashigarakami-gun (JP); Satoshi UEDA, Tokyo (JP); Ryosuke USAMI, Tokyo (JP)

(73) Assignee: FUJIFILM Corporation, Tokyo (JP)

(21) Appl. No.: 15/010,772

(22) Filed: Jan. 29, 2016

Related U.S. Application Data
(63) Continuation of application No. PCT/JP2014/068150, filed on Jul. 8, 2014.

(30) Foreign Application Priority Data
Jul. 31, 2013 (JP) 2013-159927

(51) Int. Cl. A61B 8/00; A61B 8/08

(52) U.S. Cl. CPC A61B 8/463; A61B 8/54; A61B 8/4444; A61B 8/0883; A61B 8/5223

(57) ABSTRACT

The assessment assistance device assists FAST by displaying and updating an assessment assistance screen. On the assessment assistance screen, 6 sites to be assessed through FAST are shown using hatching which is added to a schema image. In addition, execution situation display sections corresponding to these 6 sites are provided in the assessment assistance screen and are connected to the corresponding sites using indicators of arrows. A message indicating that no assessment is performed is displayed on the execution situation display sections in a state in which the corresponding sites are not assessed. When an assessment is performed in an ultrasonic assessment device by operating an assessment site designation button of an execution situation display section corresponding to a site on which an assessment is performed, the assessment assistance screen is updated by displaying an ultrasonic image which is acquired through this assessment on the execution situation display section provided with the assessment site designation button.

### CROSS-REFERENCE TO RELATED APPLICATIONS

This application is a Continuation of PCT International Application No. PCT/JP2014/068150 filed on Jul. 8, 2014, which claims priority under 35 U.S.C. §119(a) to Japanese Patent Application No. 2013-159927 filed Jul. 31, 2013. The above application is hereby expressly incorporated by reference, in its entirety, into the present application.

### BACKGROUND OF THE INVENTION

1. Field of the Invention

The present invention relates to an assessment assistance device which assists a medical assessment.

2. Description Related to the Prior Art

An assessment assistance device which assists a medical assessment, for example, guiding a method of operating a medical assessment device which performs a medical assessment is known (for example, JP2005-124712A (U.S. Pat. No. 7,806,824B), JP2004-016268A (U.S. Pat. No. 7,074,185B), and JP2008-068133A). In JP2005-124712A, JP2004-016268A, and JP2008-068133A, an ultrasonic assessment device is disclosed as a medical assessment device and an assessment assistance function is incorporated in the ultrasonic assessment device.

The ultrasonic assessment device has an ultrasonic probe for obtaining an ultrasonic image. The ultrasonic assessment device generates an ultrasonic image of an assessment site of a subject by applying the ultrasonic probe to the assessment site and displays the image. In some cases, a skill is required for the method (position, direction, or the like) of applying the ultrasonic probe thereto depending on the assessment site. For this reason, ultrasonic assessment devices disclosed in JP2005-124712A, JP2004-016268A, and JP2008-068133A are provided with an assessment assistance screen display function which displays an assessment assistance screen which is related to the method of applying an ultrasonic probe to a display.

In the assessment assistance screen disclosed in JP2005-124712A, a position guide image in which the position of an assessment site is marked in a schematic view (schema) of the human body is displayed and a guidance of the direction in which the ultrasonic probe is applied is displayed. In the assessment assistance screens disclosed in JP2004-016268A and JP2008-068133A, a guidance of the direction in which the ultrasonic probe is applied is displayed.

In all of the assessment assistance screens disclosed in JP2005-124712A, JP2004-016268A, and JP2008-068133A, the guidance of the position or the direction of one assessment site is displayed.

In recent years, a prompt and easy ultrasonography using an ultrasonic assessment device is performed in order to promptly and easily perform initial diagnosis of a trauma. This prompt and easy ultrasonography is called focused assessment with sonography for trauma (FAST). FAST is performed in order to perform an ultrasonic assessment on 6 assessment sites including a pericardial cavity (a space between the heart and an outer membrane which covers the heart), the right and left intercostals, the Morison's pouch (a region existing between the liver and the right kidney), the Douglas' pouch (a part of peritoneal cavity existing between the uterus and the rectum), and the periphery of the spleen and to check the presence and absence of a large amount of hemothorax caused by a trauma to the abdominal region, intraperitoneal bleeding, cardiac tamponade (a state in which pulsation of the heart is inhibited due to a large amount of fluid stored between the heart and the pericardium), or the like. Moreover, FAST is performed as a primary assessment in order to determine a treatment policy, such as the necessity of an abdominal operation, after checking the presence and absence of fluid storage (bleeding) in the 6 sites.

FAST is used in the primary assessment, and therefore, it is preferable to perform FAST as early as possible after a patient is injured. For this reason, in some cases, FAST is performed by a rescue worker at the scene of the accident or a transport vehicle such as an ambulance while a patient is taken to the hospital from the scene of the accident, or the like. Since a rescue worker is not skilled in an assessment procedure as much as a physician, the assessment assistance devices disclosed in JP2005-124712A, JP2004-016268A, and JP2008-068133A, are effective for the rescue worker.

However, in a case where the assessment assistance devices disclosed in JP2005-124712A, JP2004-016268A, and JP2008-068133A are used to perform assessments, such as FAST, continuously on a plurality of assessment sites which have been determined in advance, it is difficult to use the assessment assistance devices, and therefore, there is room for improvement.

It is important to accurately guide the positions of the assessment sites when performing assessments continuously on a plurality of assessment sites which have been determined in advance. However, it is also important to prevent an omission of an assessment of a part of the plurality of assessment sites, for example, forgetting an assessment by accident. Particularly, a prompt response is required in an emergency medical field, and therefore, the omission of an assessment easily occurs. Thus, a countermeasure to prevent the omission of an assessment has been required.

In the assessment assistance devices disclosed in JP2005-124712A, JP2004-016268A, and JP2008-068133A, there is a disclosure of an assessment assistance screen for guiding the method (position, direction, or the like) of applying an ultrasonic probe on each assessment site. However, there is neither an indication nor suggestion of the tasks such as prevention of an omission of an assessment or the configuration for solving the problems.

### SUMMARY OF THE INVENTION

The present invention has been made in consideration of the above-described problems, and an object of the invention is to provide an assessment assistance device which can prevent the omission of an assessment when performing assessments continuously on a plurality of assessment sites.

In order to achieve the above-described purpose, the assessment assistance device of the present invention for assisting a medical assessment includes an execution situation reception unit and a display control unit. The execution situation reception unit receives an input of an execution situation indicating whether or not assessments are executed on a plurality of assessment sites which have been determined in advance. The display control unit displays an assessment assistance screen including a schema image and an execution situation display section on a display. In the schema image, positions of the plurality of assessment sites are marked on a schematic view of a human body. The execution situation display section is provided corresponding to each of the assessment sites within the schema image, and displays the execution situation. The display control unit updates display contents of the execution situation display section based on the execution situation which the execution situation reception unit has received.

The medical assessment may be performed using a medical device that outputs an assessment result.

The execution situation reception unit is adapted to receive the assessment result as the execution situation, and the display control unit may display the assessment result on the execution situation display section corresponding to an assessment site in which an assessment is executed, out of a plurality of the execution situation display sections.

The execution situation display section may display whether or not the assessment is executed using presence and absence of the display of the assessment result.

The display control unit may display information indicating that no assessment has been performed, on the execution situation display section corresponding to an assessment site in which no assessment has been executed, out of a plurality of the execution situation display sections.

In the case where the order of executing assessments on the plurality of assessment sites is determined, a number indicating the order thereof may be displayed on the execution situation display section.

The display control unit may display the assessment assistance screen in a state that all of the plurality of assessment sites and the plurality of the execution situation display sections corresponding to the plurality of assessment sites are viewable, on the display.

A medical device may be an ultrasonic assessment device which has an ultrasonic probe and is adapted to generate an ultrasonic image based on a signal detected by the ultrasonic probe and output the ultrasonic image as an assessment result, and the assessment sites may be sites with which the ultrasonic probe comes into contact.

The assessment assistance device may be used at the time of performing FAST that is prompt and easy ultrasonography using the ultrasonic assessment device.

The plurality of assessment sites may be 6 sites including pericardial cavity, Morison's pouch, right thoracic cavity, periphery of the spleen, left thoracic cavity, and Douglas' pouch which are determined in advance, in FAST.

A distribution unit for distributing the assessment assistance screen to a predetermined distribution destination may be provided.

A reception unit for receiving assistance information relating to the assessments which have been sent from the predetermined distribution destination may be provided.

In the present invention, it is possible to confirm whether each assessment site is assessed while glancing at the assessment assistance screen by displaying the assessment assistance screen which includes a schema image, in which an assessment site is marked, and, the execution situation display section which displays an execution situation indicating whether or not an assessment of each of the assessment sites is executed. Therefore, it is possible to provide an assessment assistance device which can prevent an assessment omission when continuously performing assessments on the plurality of assessment sites.

### BRIEF DESCRIPTION OF DRAWINGS

For more complete understanding of the present invention, and the advantage thereof, reference is now made to the subsequent descriptions taken in conjunction with the accompanying drawings, in which:

FIG. 1 is a schematic view showing a configuration of a medical assessment assistance system;

FIG. 2 is a schematic block diagram of an assessment assistance device;

FIG. 3 is a view illustrating the function of the assessment assistance device;

FIG. 4 is a flowchart showing an order of displaying and updating an assessment assistance screen;

FIG. 5 is an explanatory view showing an assessment assistance screen in an initial state;

FIG. 6 is an explanatory view showing an assessment assistance screen after update;

FIG. 7 is an explanatory view showing an example in which a help screen is stored in a storage device;

FIG. 8 is an explanatory view showing an example in which help buttons are arranged on the assessment assistance screen.

FIG. 9 is an explanatory view showing an example of a help screen;

FIG. 10 is an explanatory view showing a configuration of a system which transmits an assessment assistance screen to a management center and receives assessment assistance information from the management center;

FIG. 11 is an explanatory view showing an example in which the assessment assistance device is provided with a function as a communication unit;

FIG. 12 is an explanatory view showing a configuration of a system which performs an assessment through a remote operation from the management center; and

FIG. 13 is an explanatory view showing an example in which an assessment request buttons are arranged on the assessment assistance screen.

### DESCRIPTION OF THE PREFERRED EMBODIMENTS

### First Embodiment

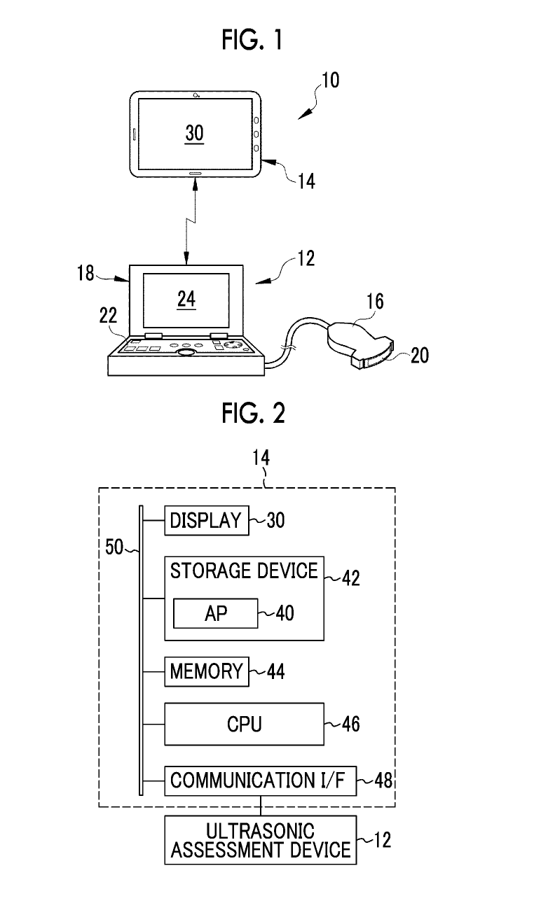

In FIG. 1, a medical assessment assistance system 10 includes an ultrasonic assessment device (medical device) 12 and an assessment assistance device 14. The assessment assistance device 14 and the ultrasonic assessment device 12 are installed in, for example, an ambulance and are connected to each other so as to be communicable through a wired system or a wireless system.

The ultrasonic assessment device 12 has an ultrasonic probe 16 and a main body 18. The ultrasonic probe 16 is connected to the main body 18. The ultrasonic probe 16 is used by being brought into contact with the body of a patient as is well known. A distal end of the ultrasonic probe 16 is provided with an ultrasonic transducer (UT) array 20. The UT array 20 is controlled by the main body 18 and emits an ultrasonic wave when a drive signal is input from the main body 18. In addition, the UT array 20 detects a reflection wave of the emitted ultrasonic wave and outputs a detection signal to the main body 18.

The main body 18 has an operating unit 22 and a monitor 24, drives the UT array 20 based on an operation instruction which has been input through an operation of an operating unit 22, and emits an ultrasonic wave from the UT array 20. In addition, an ultrasonic image is generated based on the detection signal which has been input from the UT array 20 in accordance with the emission of an ultrasonic wave, and the generated ultrasonic image is displayed on the monitor 24. The emission of an ultrasonic wave and the output of a detection signal are repeated at a predetermined cycle (for example, 10 times per second) and ultrasonic images are repeatedly generated in accordance with this cycle and are updated and displayed on the monitor 24.

In a case of using the ultrasonic assessment device 12, first, the ultrasonic probe 16 is pressed by an assessment site in a posture in which the UT array 20 and the assessment site which becomes an assessment target are opposed to each other. An ultrasonic image is displayed in accordance with the assessment site pressed by the ultrasonic probe 16 is displayed on the monitor 24. The position, the angle, or the like of the ultrasonic probe 16 is adjusted based on the display of the ultrasonic image on the monitor 24. It is possible to store the ultrasonic image which been displayed on the monitor 24. When a storage instruction is input through the operating unit 22 at a predetermined timing, an ultrasonic image which is being displayed at that time is once recorded in an internal memory (not shown) of the main body 18. The ultrasonic image which has been recorded in the internal memory is transmitted to the assessment assistance device 14. The ultrasonic image may be stored in the assessment assistance device 14 or in an image server which is connected to the assessment assistance device 14.

An exclusive operation button for transmitting the ultrasonic image to the assessment assistance device 14 may be provided in the operating unit 22 to transmit the ultrasonic image to the assessment assistance device 14 in response to the operation of the operation button. In addition, the ultrasonic image to be stored is not limited to be in a form of a static image, and may be in a form of a moving image.

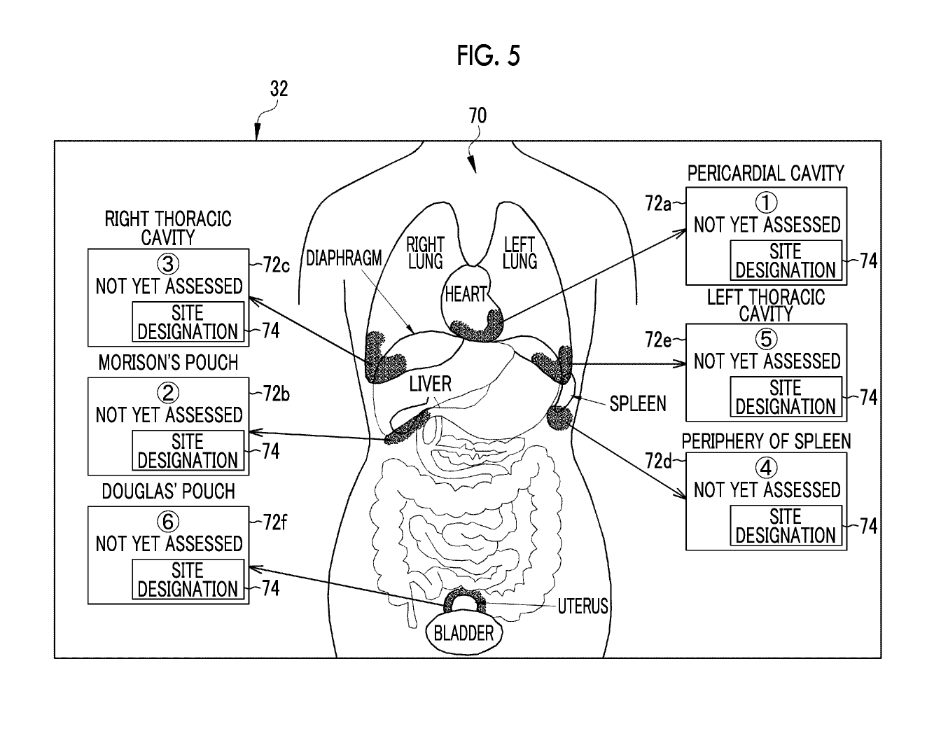

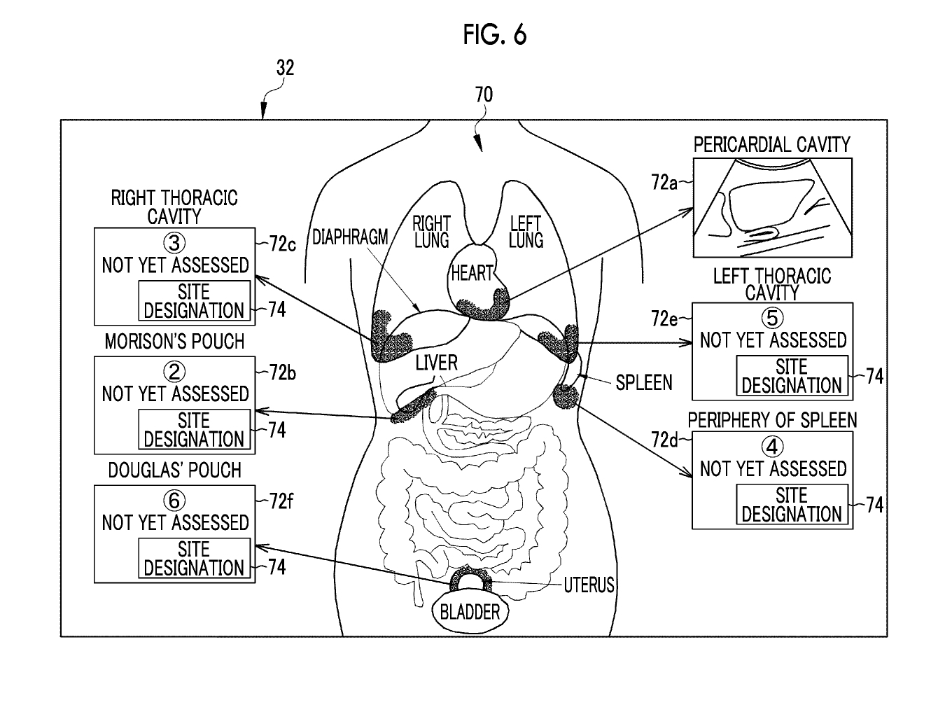

The assessment assistance device 14 has, for example, a touch panel type display 30 which is a tablet type computer. The assessment assistance device 14 can input various operation instructions through a touch operation on the display 30. In addition, the assessment assistance device 14 displays an assessment assistance screen 32 (refer to FIGS. 5 and 6) on the display 30 and assists assessments which are continuously executed on a plurality of assessment sites by updating the assessment assistance screen 32 which has been displayed on the display 30. In the present embodiment, an example in which assessments which are continuously executed on the plurality of assessment sites are FAST will be described.

As shown in FIG. 2, the assessment assistance device 14 is constituted based on a tablet type computer by installing a control program such as an operating system, or an application program (AP) 40 for causing a computer to function as the assessment assistance device 14.

The assessment assistance device 14 includes a storage device 42, a memory 44, a central processing unit (CPU) 46, and a communication interface (I/F) 48 in addition to the display 30, and these are connected to each other through a data bus 50. The storage device 42 is, for example, a hard disk drive and is an internal storage which is incorporated in a main body of the assessment assistance device 14. In the storage device 42, a control program, the AP 40, and data for display such as various images or messages constituting the assessment assistance screen 32 are stored.

The memory 44 is a work memory for the CPU 46 executing processing. The CPU 46 comprehensively controls each unit of the computer by loading the control program which has been stored in the storage device 42, in the memory 44 and executing processing in accordance with the program. The communication I/F 48 is an interface in which a transmission control between the communication I/F 48 and the ultrasonic assessment device 12 is performed. The assessment assistance device 14 communicates with the ultrasonic assessment device 12 through the communication I/F 48.

The AP 40 is a program for causing a computer to execute an assistance function for assisting FAST. The CPU 46 comprehensively controls each unit of the computer by loading the AP 40 which has been stored in the storage device 42 in the memory 44 and executing processing in accordance with the program.

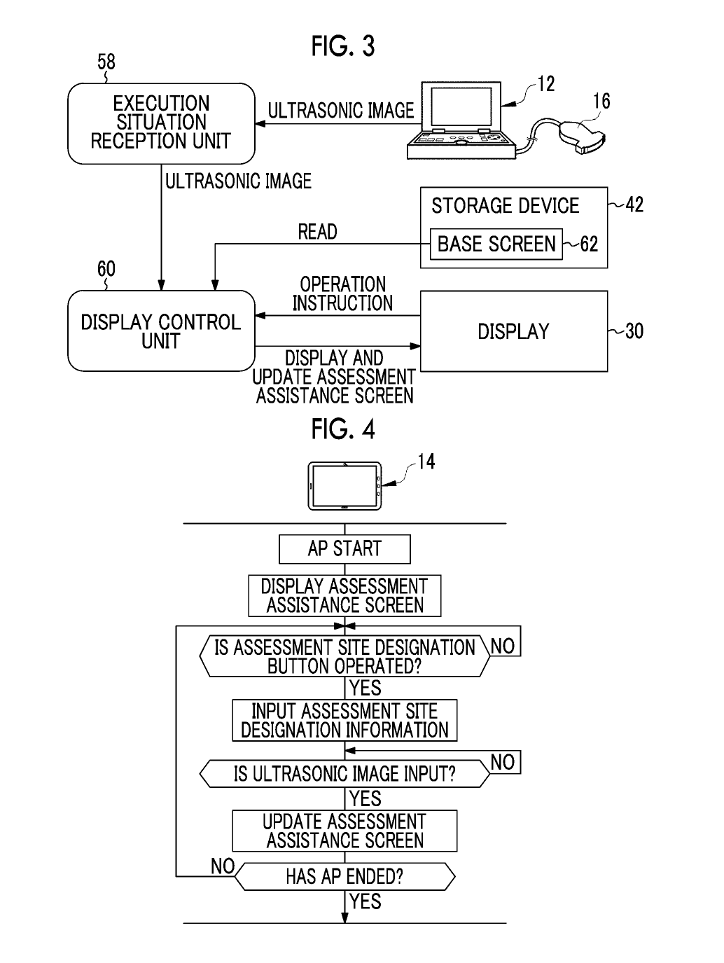

As shown in FIG. 3, when the AP 40 is started, the CPU 46 functions as an execution situation reception unit 58 and a display control unit 60 to perform assistance processing for assisting FAST in cooperation with the memory 44. In the assistance processing, the execution situation reception unit 58 receives an input of an execution situation indicating whether or not an assessment is executed. In this example, when an assessment is executed using the ultrasonic assessment device 12, the execution situation reception unit 58 receives an ultrasonic image, which has been sent from the ultrasonic assessment device 12, as an execution situation indicating whether or not an assessment is executed. That is, the ultrasonic image is an assessment result of an ultrasonic assessment and is information which cannot be obtained if an assessment is not executed. For this reason, an assessment is not executed if there is no input of an ultrasonic image, and an assessment is executed if there is an input of an ultrasonic image. The input of an ultrasonic image means an input of an execution situation.

The execution situation reception unit 58 receives an input of an execution situation by receiving an ultrasonic image which has been sent from the ultrasonic assessment device 12. The received ultrasonic image is output to the display control unit 60. In addition, in the assistance processing, the display control unit 60 displays the assessment assistance screen 32 on the display 30. Moreover, the display control unit 60 updates the display contents of execution situation display sections 72a to 72f within the assessment assistance screen 32 based on the ultrasonic image which has been sent from the ultrasonic assessment device 12.

When the assistance processing is started in accordance with the AP 40 as shown in FIG. 4, the display control unit 60 displays the assessment assistance screen 32 on the display 30. A base screen 62 (refer to FIG. 3) which becomes a base of the assessment assistance screen 32 is stored in the storage device 42, the display control unit 60 reads the base screen 62 from the storage device 42, and the read base screen 62 is displayed in the display 30 as an assessment assistance screen 32 in an initial state.

As shown in FIG. 5, a schema image 70 and execution situation display sections 72a to 72f are arranged on an assessment assistance screen 32 (that is, base screen 62) in an initial state which is first displayed in accordance with the start of the AP 40. The schema image 70 is a schematic view in which anatomical features of the human body is schematically displayed, and the positions of the 6 assessment sites including the “pericardial cavity”, the “Morison's pouch”, the “right thoracic cavity”, the “periphery of the spleen”, the “left thoracic cavity”, and the “Douglas' pouch”, which are to be assessed by FAST, are marked on the schematic view of the human body using hatching.

The execution situation display sections 72a to 72f correspond to either of the 6 assessment sites to be assessed by FAST. In the assessment assistance screen 32, the execution situation display sections 72a to 72f are respectively connected to corresponding assessment sites using the indicators of arrows in order to show the corresponding relationship therebetween. In addition, in the execution situation display sections 72a to 72f, numbers (1) to (6) showing the order to be assessed, messages of “not yet assessed” which indicate that no assessment has been performed, and assessment site designation buttons 74 (which are disclosed as “site designation” in the drawing) for designating an assessment site in the assessment assistance screen 32 when performing an assessment are arranged.

Returning to FIG. 4, after displaying the assessment assistance screen 32 in an initial state, the display control unit 60 enters an operation standby state of the assessment site designation buttons 74. Then, when any of the assessment site designation buttons 74 in the execution situation display sections 72a to 72f are operated during the operation standby state, operation information indicating that one assessment site designation button 74 out of the plurality of assessment site designation buttons 74 is operated is input to the display control unit 60. The assessment site designation buttons 74 which are respectively provided in the execution situation display sections 72a to 72f correspond to the assessment sites. Therefore, each of the respective operation information pieces of the assessment site designation buttons 74 functions as assessment site designation information relating to an assessment site corresponding to an assessment site designation button 74 which has been operated. For this reason, the assessment site designation information is input to the display control unit 60 and an assessment site is designated through the operation of the assessment site designation button 74. The method of designating an assessment site, for example, designating an assessment site by touching an execution situation display section corresponding to the assessment site instead of designating the assessment site by operating an assessment site designation button 74, can be appropriately changed.

The display control unit 60 enters an input standby state to wait for an input of an ultrasonic image relating to the designated assessment site from the ultrasonic assessment device 12, when the assessment site designation information is input to the display control unit. Then, when the ultrasonic image is sent from the ultrasonic assessment device 12 to the assessment assistance device 14, the ultrasonic image is input to the display control unit 60 through the execution situation reception unit 58. The display control unit 60 updates the assessment assistance screen 32 by displaying the input ultrasonic image on the execution situation display section corresponding to the designated assessment site.

For example, when an assessment site designation button 74 of an execution situation display section 72a corresponding to the “pericardial cavity” is operated in a state in which the assessment assistance screen 32 in an initial state shown in FIG. 5 is displayed, assessment site designation information which designates the “pericardial cavity” as an assessment site is input to the display control unit 60. When an ultrasonic image is sent from the ultrasonic assessment device 12 to the assessment assistance device 14 after the assessment site is designated, the display control unit 60 updates the assessment assistance screen 32 in an initial state in which ultrasonic images of the execution situation display sections 72a to 72f are not displayed to an assessment assistance screen 32 shown in FIG. 6 which displays an ultrasonic image of the “pericardial cavity” on the execution situation display section 72a.

Returning to FIG. 4, after updating the assessment assistance screen 32, the display control unit 60 enters an operation standby state of the assessment site designation buttons 74 again, and repeats the updating of assessment assistance screens 32 in the above-described order until the AP 40 ends.

In this manner, in the assessment assistance screen 32, assessment sites to be assessed are marked using hatching which is added to the schema image 70 in FAST, and therefore, it is possible to grasp the assessment sites at a glance. In addition, in the assessment assistance screen 32, an ultrasonic image is displayed on an execution situation display section corresponding to an assessment site which has been assessed, as an assessment result, and a message which indicates that no assessment has been performed is displayed on an execution situation display section corresponding to an assessment site which has not been assessed. Therefore, it is possible to grasp the assessment site which has been assessed and the assessment site which has not been assessed at a glance. For this reason, it is possible to check whether or not an assessment has been performed on each assessment site at a glance even in a case where assessments are continuously performed on a plurality of assessment sites, and thus, it is possible to prevent an omission of an assessment.

The message using a letter of “not yet assessed” may not be displayed. For example, whether or not an assessment is executed may be displayed in each of the execution situation display sections 72a to 72f depending on the presence and absence of the display of the ultrasonic image as the assessment result.

Furthermore, in the assessment assistance screen 32, a number indicating the order of assessments is given to an execution situation display section corresponding to an assessment site which has not been assessed, that is, an assessment site to be assessed. Therefore, it is also possible to grasp the order of assessments at a glance. In this example, the number one is the “pericardial cavity”, the number two is the “Morison's pouch”, the number three is the “right thoracic cavity”, the number four is the “periphery of the spleen”, the number five is the “left thoracic cavity”, and the number six is the “Douglas' pouch”.

The size of the image of the assessment assistance screen 32 can be freely set. Therefore, the size of the image of the assessment assistance screen 32 may be set to be larger than that of the screen of the display 30. In this case, the entirety of the assessment assistance screen 32 cannot be displayed once on the display 30, and therefore, it is necessary to scroll the screen of the display in order to display a hidden part. However, in some cases, it is impossible to grasp the situations of assessments of sites (whether or not assessments have been performed) at a glance depending on the size of the image of the assessment assistance screen 32. For this reason, it is preferable to adjust the size of the image of the assessment assistance screen 32 to the size of the screen of the display 30 such that the entirety of the assessment assistance screen 32 fits within the screen of the display 30. In this manner, all execution situation reception units can be viewed, and therefore, it is possible to grasp the execution situations of the assessment sites. As a matter of course, the assessment assistance screen 32 may be displayed by being enlarged or reduced based on an enlargement/reduction operation which is received from a user. All of the execution situation reception units may be viewed, and the entirety of the assessment assistance screen 32 may not always fit within the screen of the display 30.

In addition, in this example, a mode of using an input of an ultrasonic image has been described as an input of an execution situation of an assessment, but information different from the ultrasonic image may be used. For example, an ultrasonic image and an assessment completion signal may be sent together from the ultrasonic assessment device 12 to the assessment assistance device 14 and the execution situation reception unit 58 may receive the sent assessment completion signal as an execution situation. In this case, when an assessment designation button 74 is operated in the assessment assistance device 14, the operation signal is sent to the ultrasonic assessment device 12. Accordingly, the ultrasonic assessment device 12 is notified that the assessment assistance device 14 waits for an input of an assessment completion signal. After the notification is received, the ultrasonic assessment device 12 sends the ultrasonic image and the assessment completion signal together when sending the ultrasonic image to the assessment assistance device 14. A signal indicating that an assessment has not been completed may be periodically sent to the assessment assistance device 14 as a signal indicating an execution situation while the assessment completion signal is sent thereto.

### Second Embodiment

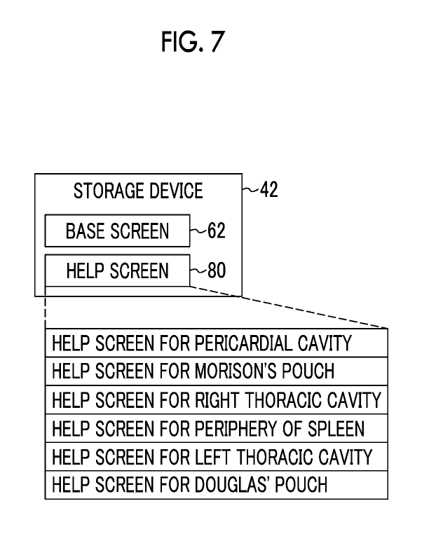

Subsequently, a second embodiment of the present invention will be described using FIGS. 7 to 9. In the following description, the same members as those in the above-described embodiment are given the same reference numerals, and the description thereof will not be repeated.

As shown in FIG. 7, in the second embodiment, a help screen 80 is stored in the storage device 42 in addition to the base screen 62. Six kinds of help screens 80 including a “help screen for the pericardial cavity”, a “help screen for the Morison's pouch”, a “help screen for the right thoracic cavity”, a “help screen for the periphery of the spleen”, a “help screen for the left thoracic cavity”, and a “help screen for the Douglas' pouch” corresponding to all six assessment sites to be assessed in FAST. Each of the help screens 80 is a screen in which matters (such as the position or the posture (direction facing the UT array 20) of the ultrasonic probe 16) that require attention while inspecting a corresponding assessment site, using letters, diagrams, photographs, or the like.

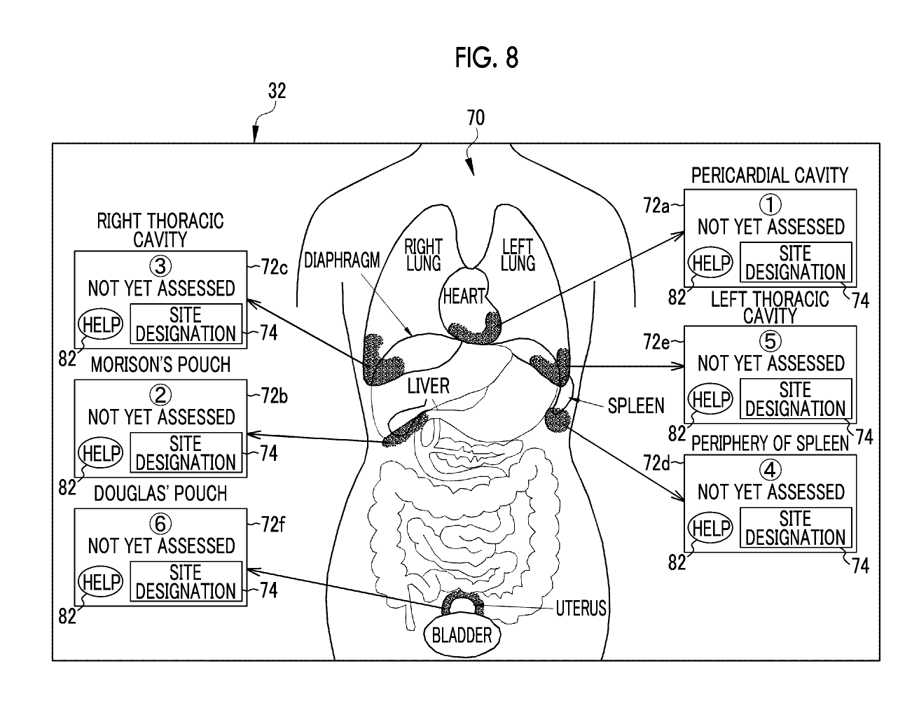

As shown in FIG. 8, in the assessment assistance screen 32 in the second embodiment, a help button 82 is provided in an execution situation display section corresponding to an assessment site, which has not been assessed, out of the execution situation display sections 72a to 72f in addition to an assessment site designation button 74. Moreover, in the second embodiment, when the help button 82 is operated, operation information indicating that one help button 82 out of a plurality of help buttons is operated is input to the display control unit 60. The help buttons 82 which are respectively provided in the execution situation display sections 72a to 72f correspond to assessment sites. Therefore, operation information of each of the help buttons 82 functions as assessment site designation information relating to an assessment site corresponding to an operated help button 82. For this reason, assessment site designation information is input to the display control unit 60 by operating the help button 82 and an assessment site is designated. When the display control unit 60 receives the assessment site designation information through an operation of the help button 82, the display control unit reads a help screen 80 of the designated assessment site from the storage device 42 to display the help screen on the display 30.

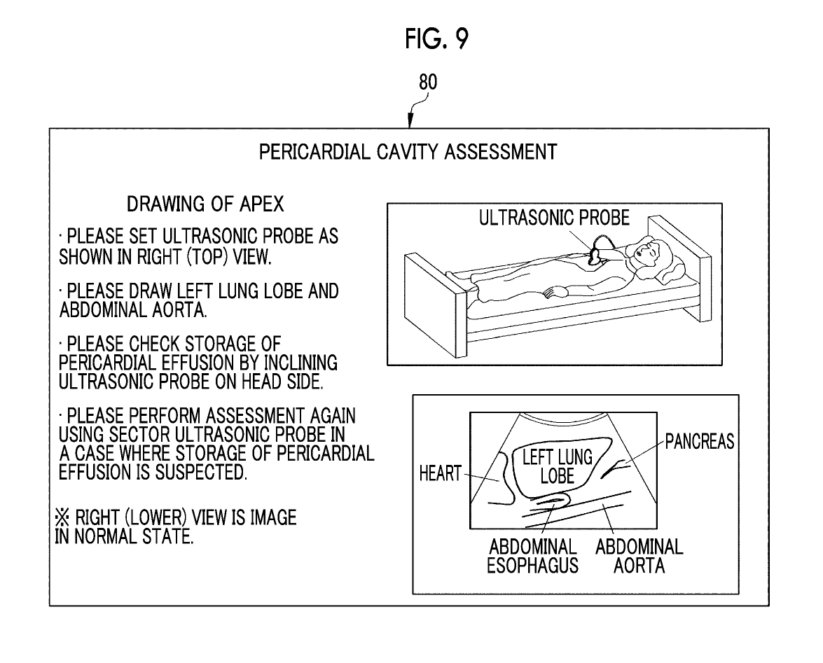

For example, when a help button 82 which is provided in an execution situation display section 72a corresponding to the “pericardial cavity” is operated in a state in which the assessment assistance screen 32 shown in FIG. 5 is displayed, a help screen 80 which corresponds to an assessment of the “pericardial cavity” and is shown in FIG. 9 is displayed on the display 30.

In this manner, in a case of desiring to refer to a help screen on the assessment assistance screen 32 on which assessment sites to be assessed are listed, it is possible to directly display a help screen 80 of each of the assessment sites from the assessment assistance screen 32 by operating an help button 82 corresponding to the assessment site to be referred to. For this reason, it is possible to simply display a desired help screen in comparison with a case of searching a help screen by, for example, inputting the name or the like of the assessment site to be referred to, and designating the help screen to be displayed from the searched list.

The display mode of the help screens 80 in the second embodiment can be freely set. Therefore, the help screen 80 and the assessment assistance screen 32 may be displayed at the same time by, for example, displaying the help screen 80 on the assessment assistance screen 32 in an overlapping manner. In addition, either of the help screen 80 or the assessment assistance screen 32 may be displayed by, for example, displaying the help screen 80 instead of the assessment assistance screen 32.

### Third Embodiment

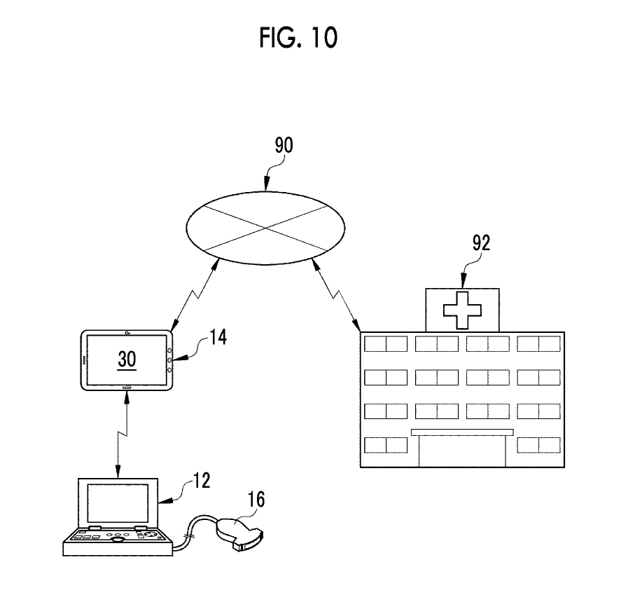

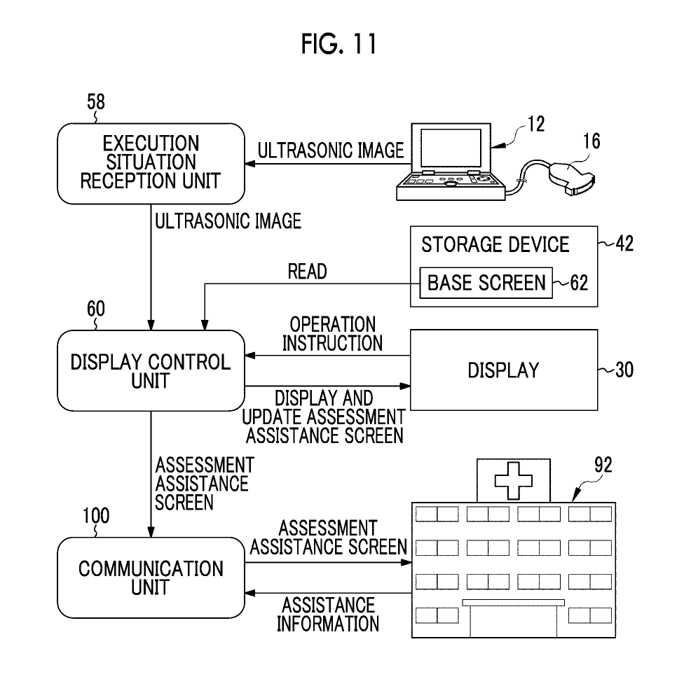

Next, a third embodiment of the present invention will be described using FIGS. 10 and 11. As shown in FIG. 10, in the third embodiment, the assessment assistance device 14 is connected to a management center 92 through a communication network 90 such as the Internet or a local area network (LAN). The management center 92 is, for example, a general hospital which manages regional medical care. Various kinds of information pieces can be transmitted and received between the assessment assistance device 14 and the management center 92 through a communication I/F 48 and the communication network 90.

As shown in FIG. 11, in the third embodiment, the CPU 46 cooperates with the memory 44 in accordance with the start of the AP 40 and functions as the display control unit 60 and a communication unit (a distribution unit or a reception unit) 100. The display control unit 60 displays and updates an assessment assistance screen 32 as described above and outputs a displayed or updated assessment assistance screen 32 to the communication unit 100 every time when the display and the update of the assessment assistance screen 32 is performed in this manner. The communication unit 100 distributes the assessment assistance screen 32 which has been input from the display control unit 60 to the management center 92.

In the management center 92, it is possible to refer to the distributed assessment assistance screen 32 by displaying the assessment assistance screen on a monitor (not shown) within the management center 92, or the like. In addition, in the management center 92, assistance information such as an advice based on the distributed assessment assistance screen 32 can be transmitted to the assessment assistance device 14. The communication unit 100 also receives the assistance information which has been sent from the management center 92 in this manner. The assistance information may be sent in a form of electronic data such as an electronic mail, or may be transmitted through voice communication using a voice communication function possessed by the assessment assistance device 14.

In the third embodiment, it is possible to observe the common assessment assistance screen 32 in both of the assessment assistance device 14 and the management center 92 in this manner, and therefore, it is possible to grasp the progress status of FAST or the assessment result of each of the units in the management center 92 in real time. In addition, in the third embodiment, assistance information relating to FAST can be transmitted from the management center 92 to the assessment assistance device 14, and therefore, it is possible to perform more satisfactory assistance.

### Fourth Embodiment

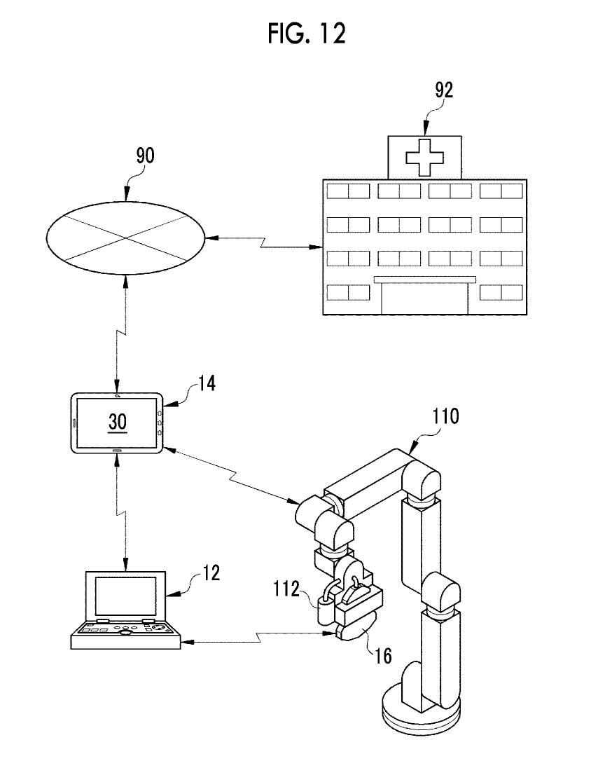

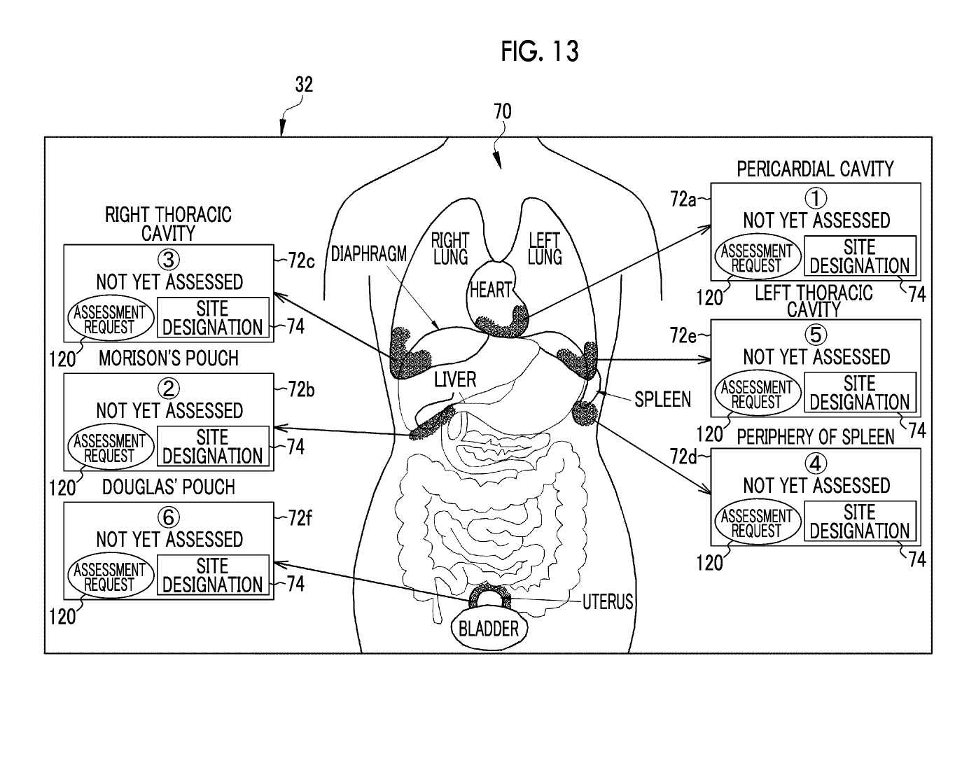

Subsequently, a fourth embodiment of the present invention will be described using FIGS. 12 and 13. As shown in FIG. 12, in the fourth embodiment, the ultrasonic assessment device 12 is driven and controlled by the assessment assistance device 14. The ultrasonic assessment device 12 generates an ultrasonic wave from the ultrasonic probe 16 based on the control of the assessment assistance device 14, generates an ultrasonic image based on a reflection wave of the generated ultrasonic wave, and transmits the generated ultrasonic image to the assessment assistance device 14.

In addition, in the fourth embodiment, the ultrasonic probe 16 is fixed to a distal portion of a robot arm 110. A proximal portion of the robot arm 110 is fixed to a predetermined position (such as the floor of an ambulance), and the ultrasonic probe 16 can be moved to a predetermined position or posture by operating a plurality of joints. The robot arm 110 is connected to the assessment assistance device 14 and is operated by being driven and controlled by the assessment assistance device 14.

A camera 112 is attached to a distal end of the robot arm 110. The camera 112 is also connected to the assessment assistance device 14. The camera 112 performs photographing by being controlled by the assessment assistance device 14 and outputs a photographed image to the assessment assistance device 14.

The assessment assistance device 14 is connected to the management center 92 through the communication network 90 and the image photographed by the camera is sent from the assessment assistance device 14 to the management center 92 through the communication network 90. A controller (not shown) for operating the ultrasonic assessment device 12 and the robot arm 110 is provided in the management center 92. When the controller is operated, an operation signal is input from the management center 92 to the assessment assistance device 14. The assessment assistance device 14 drives and controls the ultrasonic assessment device 12 and the robot arm 110 based on the operation signal which has been sent from the management center 92.

As shown in FIG. 13, in the fourth embodiment, an assessment request button 120 is provided in an execution situation display section corresponding to an assessment site of the assessment assistance screen 32 which has not been assessed, in addition to the assessment site designation button 74. Moreover, in the fourth embodiment, when the assessment request button 120 is operated, an assessment request of the assessment site corresponding to the execution situation display section in which the operated assessment request button 120 is provided is transmitted from the assessment assistance device 14 to the management center 92.

In the management center 92, this assessment request is received and an assessment of the requested assessment site is performed. Specifically, the robot arm 110 and the ultrasonic assessment device 12 are controlled such that an operator (such as a physician) of the management center 92 operates the controller while observing the image (image photographed by the camera 112) which has been sent from the assessment assistance device 14, and an ultrasonic assessment is performed.

In the fourth embodiment, the example in which an assessment is performed such that the operator of the management center 92 remotely operates the robot arm 110 and the ultrasonic assessment device 12 has been described. However, the assessment assistance device 14 may automatically perform an assessment by controlling the robot arm 110 and the ultrasonic assessment device 12. In this case, an assessment executing unit for executing an assessment by controlling the robot arm 110 and the ultrasonic assessment device 12 based on an image analysis unit which analyzes an image which has been photographed by the camera 112 and an image analysis result which has been obtained using the image analysis unit may be provided in the assessment assistance device 14.

Hereinabove, the example of applying the present invention to the assessment assistance device which assists FAST has been described. However, the present invention is not limited thereto. The present invention can be widely applied to an assessment assistance device which assists a series of assessments executed in a plurality of assessment sites even if the assessments are other than FAST. In the above-described embodiments, ultrasonic images, which are obtained through FAST, that is, continuous ultrasonic assessments, are displayed on the execution situation display sections. However, in a case of applying the present invention to an assessment assistance device which assists an assessment other than FAST, assessment results obtained through this assessment may be displayed on the execution situation display sections. In addition, the information pieces (assessment results) displayed on the execution situation display sections are not limited to images, and may be letters or numerical values. In addition, an example of the ultrasonic assessment device has been described as a medical device, but a medical device other than the ultrasonic assessment device may be used.

Furthermore, in the above-described embodiments, the order of assessments, and a message which indicates that no assessment has been performed are displayed on execution situation display sections corresponding to assessment sites which have not been assessed. However, other than this, the information that no assessment has been performed may be emphasized by, for example, coloring or flickering some or all of execution situation display sections corresponding to assessment sites which have not been assessed. Furthermore, as a mode through which the order of assessment is indicated other than through a message, the order of assessments may be displayed by emphasizing an execution situation display section corresponding to an assessment site to be assessed and displaying the execution situation display section execution situation so as be distinguishable from display sections corresponding to other assessment sites which have not been assessed.

In addition, a mode of displaying an execution situation display section corresponding to an assessment site which has not been assessed may also be used for the purposes of displaying that no assessment has been performed. For example, the execution situation display section may be indicated as a black solid region without displaying anything. In this manner, the assessment results are displayed on an execution situation display section which has been assessed, and therefore, it is possible to identify whether each of the assessment sites is assessed. In the present invention, an execution situation display section corresponding to an assessment site which has been assessed and an execution situation display section corresponding to an assessment site which has not been assessed may be displayed so as to be distinguishable from each other, and therefore, it is possible to appropriately change specific display modes of each of the execution situation display sections.

Although the present invention has been fully described by the way of the preferred embodiment thereof with reference to the accompanying drawings, various changes and modifications will be apparent to those having skill in this field. Therefore, unless otherwise these changes and modifications depart from the scope of the present invention, they should be construed as included therein.

What is claimed is:

1. An assessment assistance device for assisting a medical assessment, comprising:
an execution situation reception unit for receiving an input of an execution situation indicating whether or not assessments are executed on a plurality of assessment sites which have been determined in advance; and
a display control unit for displaying an assessment assistance screen on a display and updating display contents of the execution situation display section based on the execution situation received by the execution situation reception unit, the assessment assistance screen including a schema image, in which positions of the plurality of assessment sites are marked on a schematic view of a human body, and an execution situation display section, which is provided corresponding to each of the assessment sites within the schema image and displays the execution situation.

2. The assessment assistance device according to claim 1,
wherein the medical assessment is performed using a medical device that outputs an assessment result.

3. The assessment assistance device according to claim 2,
wherein the execution situation reception unit is adapted to receive the assessment result as the execution situation, and
wherein the display control unit displays the assessment result on the execution situation display section corresponding to an assessment site in which an assessment is executed, out of a plurality of the execution situation display sections.

4. The assessment assistance device according to claim 3,
wherein the execution situation display section displays whether or not the assessment is executed using presence and absence of the display of the assessment result.

5. The assessment assistance device according to claim 1,
wherein the display control unit displays information indicating that no assessment has been performed, on the execution situation display section corresponding to an assessment site in which no assessment has been executed, out of a plurality of the execution situation display sections.

6. The assessment assistance device according to claim 1,
wherein in the case where the order of executing assessments on the plurality of assessment sites is determined, a number indicating the order thereof is displayed on the execution situation display section.

7. The assessment assistance device according to claim 1,
wherein the display control unit displays the assessment assistance screen in a state that all of the plurality of assessment sites and the plurality of the execution situation display sections corresponding to the plurality of assessment sites are viewable, on the display.

8. The assessment assistance device according to claim 2,
wherein the medical device is an ultrasonic assessment device which has an ultrasonic probe and is adapted to generate an ultrasonic image based on a signal detected by the ultrasonic probe and output the ultrasonic image as the assessment result, and
wherein the assessment sites are sites with which the ultrasonic probe comes into contact.

9. The assessment assistance device according to claim 8 which is used at the time of performing FAST that is prompt and easy ultrasonography using the ultrasonic assessment device.

10. The assessment assistance device according to claim 9,
wherein the plurality of assessment sites are 6 sites including pericardial cavity, Morison's pouch, right thoracic cavity, periphery of the spleen, left thoracic cavity, and Douglas' pouch which are determined in advance, in the FAST.

11. The assessment assistance device according to claim 1 further comprising a distribution unit for distributing the assessment assistance screen to a predetermined distribution destination.

12. The assessment assistance device according to claim 11 further comprising a reception unit for receiving assistance information relating to the assessments which have been sent from the predetermined distribution destination.
